(19) United States
(12) Patent Application Publication
(10) Pub. No.: US 2008/0214646 A1
(43) Pub. Date: Sep. 4, 2008

(54) Novel Nsaids Possessing a Nitric Oxide Donor Diazen-1-Ium-1,2-Diolate Moiety

(76) Inventors: Edward E. Knaus, Edmonton (CA); Carlos Velazquez, Frederick, MD (US)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 11/914,430

(22) PCT Filed: May 16, 2006
(86) PCT No.: PCT/US06/19115

§ 371 Date: Nov. 14, 2007

Related U.S. Application Data
(60) Provisional application No. 60/681,842, filed on May 16, 2005.
(60) Provisional application No. 60/728,364, filed on Oct. 19, 2005.

(51) Int. Cl. A61K 31/404; C07C 243/06; A61K 31/131; C07D 207/50; A61P 29/00; A61K 31/40; C07D 209/42

(52) U.S. Cl. 514/419; 564/113; 514/611; 548/557; 514/426; 548/491

(57) ABSTRACT

This invention provides a prodrug that help arthritis patients without increasing cardiovascular and gastrointestinal risk. A novel group of hybrid nitric oxide-releasing non-steroidal anti-inflammatory drugs (NO-NSAIDs), moiety attached via a one -carbon methylene spacer to the carboxylic acid group of the traditional NSAIDs aspirin, ibuprofen and indomethacin were synthesized. The ester prodrugs showed equipotent anti-inflammatory activities in vivo to that of the parent aspirin, ibuprofen and indomethacin. The simultaneous release of parent drug and nitric oxide from the NO- prodrugs constitutes a potentially beneficial property for the prophylactic prevention of thrombus formation and adverse cardiovascular events such as stroke and myocardial infarction. Data acquired in an in vivo ulcer index (UI) assay showed that this group of ester prodrugs in which no lesions were observed when compared to the parent drugs at equivalent doses. Accordingly, these hybrid NO-NSAID prodrugs possessing a diazen-1-ium-1,2-diolate moiety, represents a new approach for the rational design of anti-inflammatory drugs with reduced gastric ulcerogenicity.

This application claims the benefits of U.S. provisional application 60/728,364, filed Oct. 19, 2005, and U.S. provisional application 60/681,842, filed May 16, 2005. The contents of these preceding applications are hereby incorporated in their entireties by reference into this application.

Throughout this application, various publications are referenced. The disclosures of these publications in their entireties are hereby incorporated by reference into this application to more fully describe the state of the art to which this invention pertains.

### BACKGROUND OF THE INVENTION

This invention provides a prodrug that help arthritis patients without increasing cardiovascular and gastrointestinal risk.

The major mechanism of action by which non-steroidal anti-inflammatory drugs (NSAIDs) exhibit anti-inflammatory activity involves the inhibition of cyclooxygenase (COX)-derived prostaglandin (PG) synthesis.¹⁻⁴ PGs, in addition to being undesirable effectors of inflammatory reactions, also exert important physiological functions such as gastrointestinal cytoprotection and vascular homeostasis.⁵⁻⁷ In this regard, drugs that are more selective inhibitors of the COX-2 isozyme, relative to the COX-1 isozyme, allow the beneficial synthesis of cytoprotective PGs in the stomach in conjunction with a simultaneous inhibition of proinflammatory PG synthesis in joints. Chronic use of NSAIDs is associated with alterations in gastrointestinal integrity and function^{8,9} which results in the development of gastric ulcers.¹⁰ Thus, the gastric irritant effect of aspirin (1) can be a deterrent to its long-term use for the prophylactic prevention of adverse cardiovascular events such as stroke and myocardial infarction.^{11,12} Aspirin is a unique nonselective COX inhibitor due to its ability to acetylate the Ser530 hydroxyl group in the primary COX binding site of COX-1 and COX-2. In this regard, aspirin is a 10- to 100-fold more potent inhibitor of COX-1 relative to COX-2.¹³ Acetylation of the weakly nucleophilic OH of Ser530 by aspirin is thought to result from initial binding of its COOH to Arg120 near the mouth of the COX binding site, which positions the ortho-acetoxy moiety in close proximity to the Ser530 OH, which it acetylates. Orally administered aspirin irreversibly acetylates Ser530 of COX-1 in platelets,¹⁴ which results in a complete inhibition of platelet-derived thromboxane A2 (TxA2) biosynthesis. TxA2 is a potent platelet aggregator which also induces vasoconstriction and smooth muscle proliferation.^{15,16} However, there remains a significant risk of gastrointestinal bleeding¹⁷⁻¹⁹ due to inhibition of COX-1-mediated gastric PG synthesis even with low prophylactic doses of aspirin.²⁰⁻²³

COX-2 inhibitors are new, and in many ways, an improved class of drugs that are designed to be equally effective as traditional NSAIDS but safer. Traditional NSAIDS such as aspirin, Motrin, Aleve and other prescription drugs act by blocking the production of a family of chemicals that cause inflammation known as prostaglandins. Two enzymes appear to be crucial for the production of these prostaglandins, namely COX-1 and COX-2. Traditional NSAIDS inhibit both COX-1 and COX-2. Unfortunately, this nonselective inhibition of both COX-1 and COX-2 also inhibits prostaglandins involved in helping blood to clot, and protecting our stomach from ulcers. It is now strongly believed that this non-selective inhibition of both COX-1 and COX-2 by aspirin and related compounds is why NSAIDS carry a risk of bleeding and stomach ulcerations. A new class of drugs, namely the COX-2 inhibitors, only inhibits the enzyme involved in inflammation and leaves our physiologic housekeeping functions alone.

However, the safety of COX-2 inhibitors has been questioned. The most famous event is that a blockbuster drug from Merck Vioxx was pulled off from pharmacy shelves in 2004 after Merck's trials showed an increased risk of heart and stroke damage. The two other COX-2 inhibitors on the market Celebrex and Bextra, are under intense study for their safety. On Apr. 7, 2005, the Food and Drug Administration requested that Pfizer suspend sales of Bextra in the United States. The Food and Drug Administration is requiring all prescription anti-inflammatory arthritis medicines to provide additional information about cardiovascular and gastrointestinal risk.

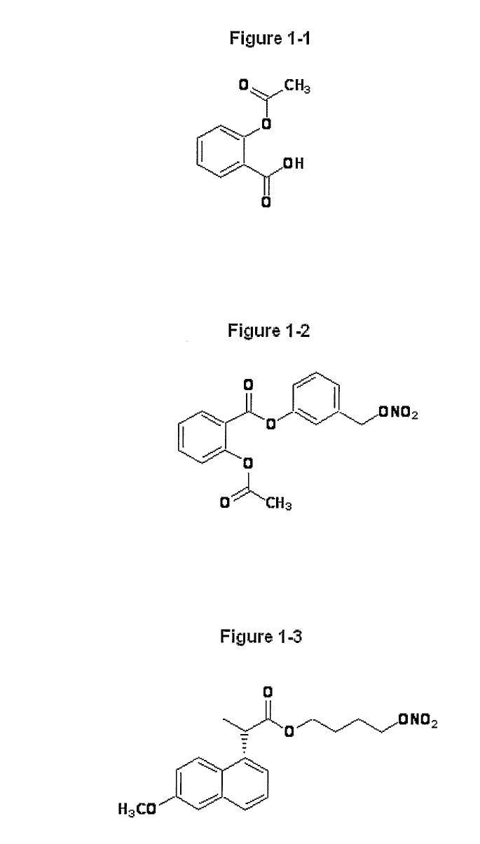

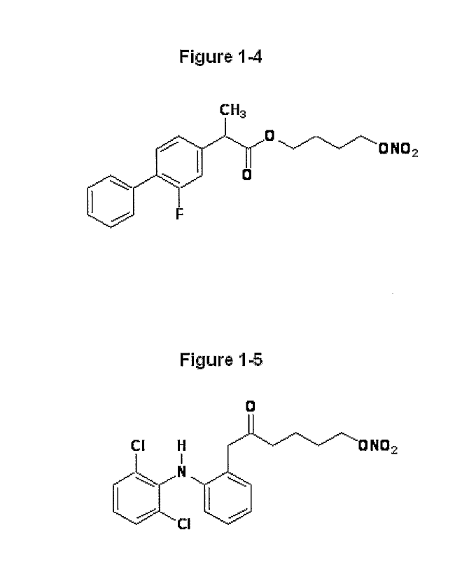

Nitric oxide (NO) is now widely recognized as a critical mediator of gastrointestinal mucosal defense, exerting many of the same actions as prostaglandins in the gastrointestinal tract.¹⁰ NO has been shown to reduce the severity of gastric injury in experimental models.^{24,25} It has been proposed that the linking of an NO-releasing moiety to an NSAID may reduce the toxicity of the latter.²⁶ In animal studies, NO-releasing derivatives of a wide range of NSAIDs (FIG. 1) including the NO-aspirin (2), NO-naproxen (3), NO-flurbiprofen (4) and NO-diclofenac (5), have been shown to spare the gastrointestinal tract, even though they suppressed prostaglandin synthesis as effectively as the parent drug.²⁶⁻³⁰ All these NO-releasing NSAIDs have a nitrooxyalkyl group as the NO-releasing group. However, an important drawback to this design is the fact that production of NO from organic nitrate esters requires a three-electron reduction, and this metabolic activation decreases in efficiency on continued use of the drugs, contributing to “nitrate tolerance”.³¹ In this regard, O²-unsubstitued N-diazen-1-ium-1,2-diolates have the potential to release up to 2 equivalents of NO with half-lives that correlate well with their pharmacological durations of action. These observations suggest that N-diazen-1-ium-1,2-diolates are minimally affected by metabolism, and are essentially different from currently available clinical vasodilators that require redox activation before NO is released.³² N-diazen-1-ium-1,2-diolates possess three attributes that make them especially attractive for designing drugs to treat a variety of disease states, namely structural diversity, dependable rates of NO release, and rich derivatization chemistry that facilitates targeting of NO to specific target organ and/or tissue sites.³² As part of our ongoing research program to develop anti-inflammatory agents with a greater safety profile, Applicants now report the synthesis, in vitro COX-1/COX-2 inhibitory activity, in vivo anti-inflammatory activity, nitric oxide release data, and results from ulcerogenicity studies for a group of ester prodrugs of aspirin, ibuprofen and indomethacin that possess a diazen-1-ium-1,2-diolate as the NO-donor moiety.

### SUMMARY OF THE INVENTION

The invention is intended to help protect chronic NSAID users such as arthritis and cardiovascular patients from potentially life-threatening gastrointestinal side effects without compromising anti-inflammatory activity. It provides a method of forming hybrid prodrugs comprising a non-steroidal anti-inflammatory drug (NSAID) linked by a methylene spacer on its carboxylic acid group to a diazen-1-ium-1,2-diolate moiety which on hydrolysis will release nitric oxide. It is intended to prevent or ameliorate gastrointestinal upset, bleeding or ulceration through the protective effect of nitric oxide in the tissues lining the gastrointestinal tract.

### DETAILED DESCRIPTION OF THE FIGURES

FIG. 1. Chemical structures of acetyl salicylic acid (1) and some representative NO-NSAIDs (organic nitrates): NO-aspirin (2), NO-naproxen (3), NO-flurbiprofen (4) and NO-dichlofenac (5).

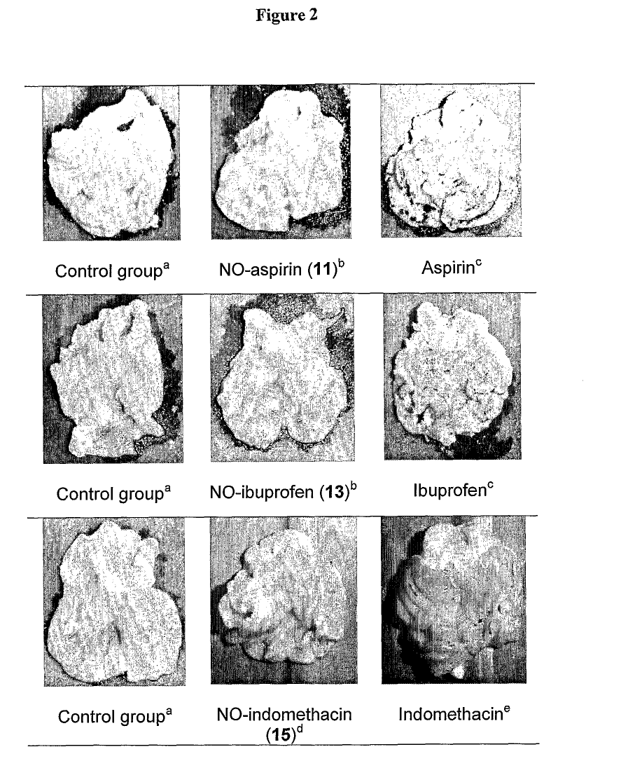

FIG. 2. Ulcerogenicity assay data illustrating the extent of NSAID-induced gastric ulcers for NO-NSAIDs 11, 13 and 15, compared to that induced by the parent drugs aspirin, ibuprofen and indomethacin.

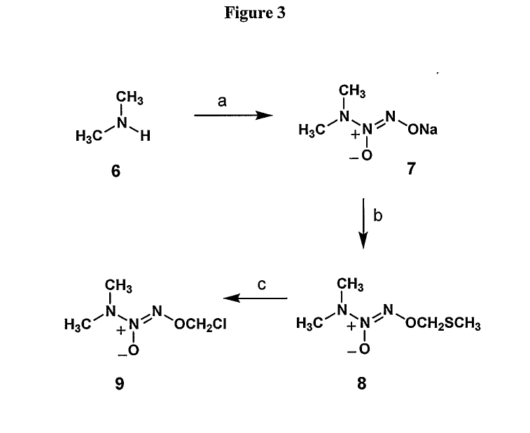

FIG. 3. O²-Chloromethyl-1-(N,N-dimethylamino)diazen-1-ium-1,2-diolate (9) preparation procedure.

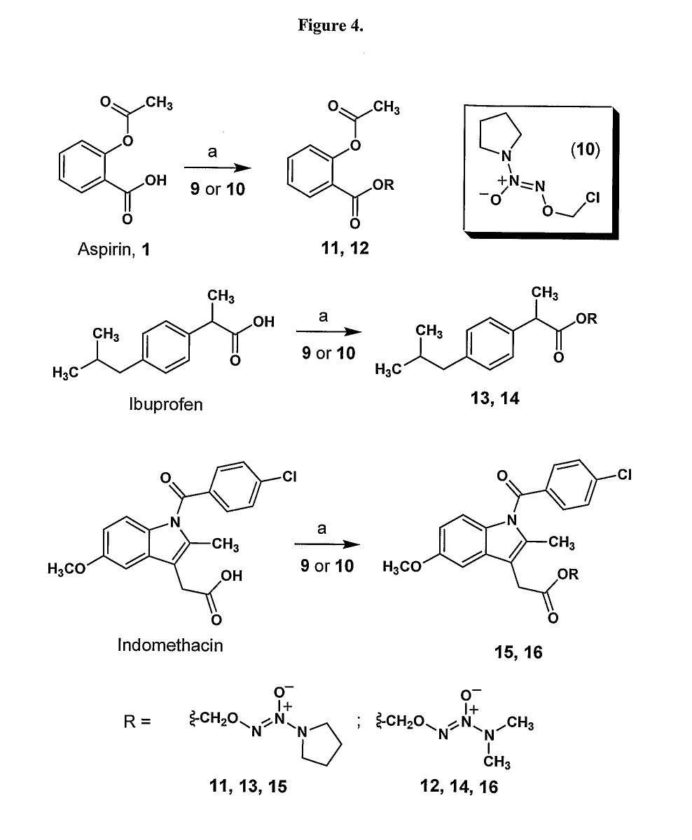

FIG. 4. Synthesis of the target NO-NSAID ester prodrugs.

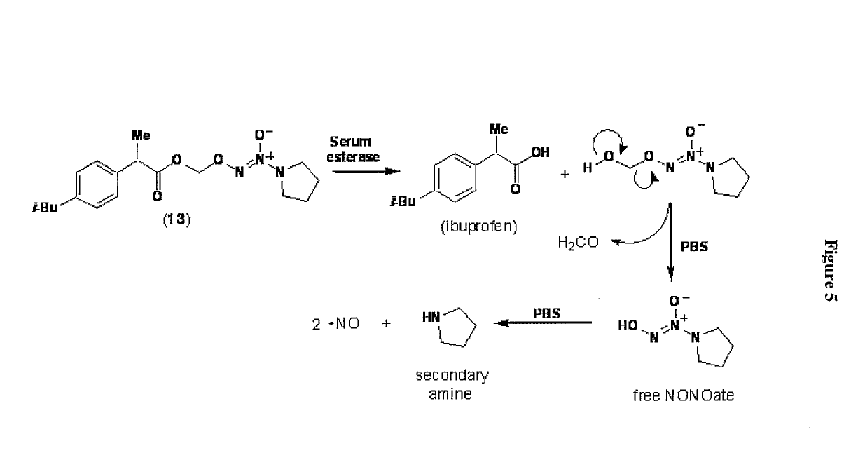

FIG. 5. Theoretical metabolic activation (hydrolysis) of NO-NSAIDs (compound 13 shown as a representative example)

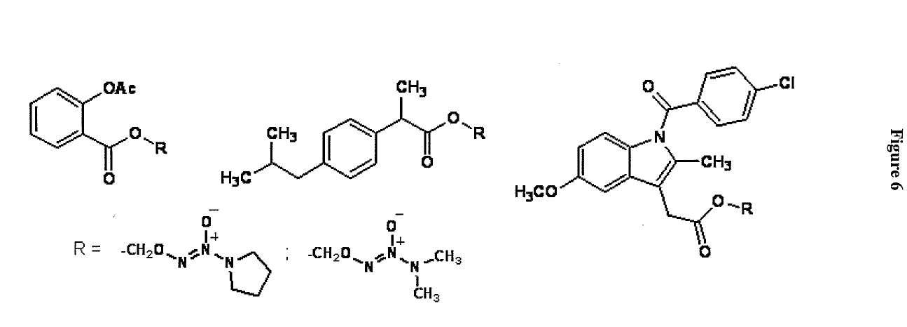

FIG. 6. Structures of new NO-releasing non-steroidal anti-inflammatory drugs based on aspirin, ibuprofen and indomethacin (NO-NSAIDs)

### DETAILED DESCRIPTION OF THE INVENTION

This invention provides a compound of the formula I:

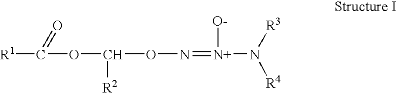

wherein R¹ is the uncarboxylated core of a non-steroidal anti-inflammatory drug, R² is hydrogen, an unsubstituted or substituted C-₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted alkoxy, nitrile, halo, an unsubstituted or substituted morpholino, amino, an unsubstituted or substituted benzyl, an unsubstituted or substituted phenyl, an unsubstituted or substituted C₁₋₄ aryl alkyl, an unsubstituted or substituted heteroaryl, an unsubstituted or substituted arylamino, an unsubstituted or substituted dialkylamino, an unsubstituted or substituted diarylamino, carboxyalkylamino, carboxydialkylamino, an unsubstituted or substituted tolyl, xylyl, anisyl, mesityl, an unsubstituted or substituted acetoxy, carboxy, an unsubstituted or substituted carboxyethyl, an unsubstituted or substituted alkylcarbonyl, thiol, an unsubstituted or substituted alkylthiol, an unsubstituted or substituted alkyloxy, carboxyamido, an unsubstituted or substituted alkylcarboxyamido, an unsubstituted or substituted dialkylcarboxyamido, an unsubstituted or substituted phenoxy, an unsubstituted or substituted benzyloxy, phenylcarbonyl, benzylcarbonyl, an unsubstituted or substituted nitrophenyl, trialkylsilyl or nitro; R³ and R⁴ are the same or different and are each preferentially one of an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted morpholino, amino, an unsubstituted or substituted benzyl, an unsubstituted or substituted C₁₋₄ aryl alkyl, an unsubstituted or substituted carboxyethyl, or the —N(R³, R⁴) group is cyclized to form a 1,2,3,4-tetrahydroquinolyl, i.e. Structure II:

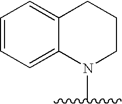

or structure III:

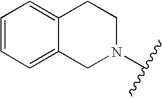

or piperidinyl, Structure IV:

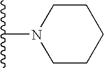

### or N-substituted-piperizinyl, Structure V:

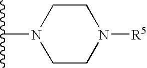

where R⁵ is an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted benzyl, an unsubstituted or substituted phenyl, an unsubstituted or substituted C₁₋₄ aryl alkyl, an unsubstituted or substituted heteroaryl, an unsubstituted or substituted tolyl, xylyl, anisyl, mesityl, an unsubstituted or substituted carboxyethyl, an unsubstituted or substituted alkylcarbonyl, phenylcarbonyl, benzylcarbonyl, an unsubstituted or substituted nitrophenyl, or trialkylsilyl.

This invention also provides a compound of the formula I, wherein the non-steroidal anti-inflammatory drug carboxylic acid in R¹ is acetylsalicylic acid, ibuprofen, naproxen, indomethacin, salicylic acid, diflunisal, salsalate, olsalazine, sulfasalazine, sulindac, etodolac, mefenamic acid, meclofenamic acid, tolmetin, ketorolac, diclofenac, fenoprofen, ketoprofen, oxaprozin, carprofen, flurbiprofen, nabumetone, any other related carboxylic acids with anti-inflammatory activity and their pharmaceutically suitable salts.

This invention provides a compound of the formula VII:

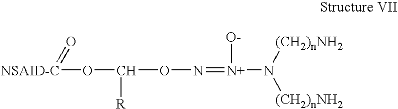

Wherein R is as in R² of Structure I, n=1−8. The structure includes pharmaceutically suitable alkali metal salts or hydrochloride salts of VII.

This invention provides a compound of Structure VIII:

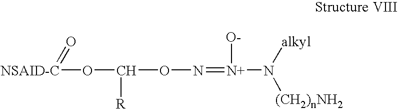

Wherein R is as in R² of Structure I, n=1−8. The structure includes pharmaceutically suitable alkali metal salts or hydrochloride salts of VIII.

This invention provides a compound of Structure IX:

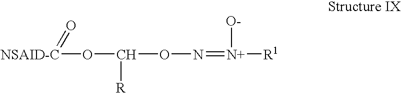

Wherein R is as in R2 of Structure I, R¹ is a N-substituted amino acid moiety.

This invention provides a compound of Structure IX above, wherein R¹ the N-substituted amino acid moiety is:

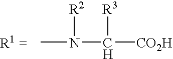

And R² is hydrogen, an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted benzyl, an unsubstituted or substituted phenyl, an unsubstituted or substituted C₁₋₄ aryl alkyl, an unsubstituted or substituted heteroaryl, an unsubstituted or substituted tolyl, xylyl, anisyl, mesityl, an unsubstituted or substituted carboxyethyl, and R³ is hydrogen, an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted alkoxy, nitrile, halo, an unsubstituted or substituted morpholino, amino, an unsubstituted or substituted benzyl, an unsubstituted or substituted phenyl, an unsubstituted or substituted C₁₋₄ aryl alkyl, an unsubstituted or substituted heteroaryl, an unsubstituted or substituted arylamino, an unsubstituted or substituted dialkylamino, an unsubstituted or substituted diarylamino, carboxyalkylamino, carboxydialkylamino, an unsubstituted or substituted tolyl, xylyl, anisyl, mesityl, an unsubstituted or substituted acetoxy, carboxy, an unsubstituted or substituted carboxyethyl, an unsubstituted or substituted alkylcarbonyl, an unsubstituted or substituted alkylthiol, an unsubstituted or substituted alkyloxy,carboxyamido, an unsubstituted or substituted alkylcarboxyamido, an unsubstituted or substituted dialkylcarboxyamido, an unsubstituted or substituted phenoxy, an unsubstituted or substituted benzyloxy, phenylcarbonyl, benzylcarbonyl, an unsubstituted or substituted nitrophenyl, trialkylsilyl or nitro. The simplest examples are N-methylglycine, N-methylalanine, N-methylphenylalanine, N-methylserine, or any other N-alkyl amino acid.

This invention provides an amide bioisostere ester compound of structure X:

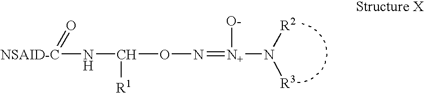

Wherein R¹ is hydrogen, an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted alkoxy, nitrile, halo, an unsubstituted or substituted morpholino, amino, an unsubstituted or substituted benzyl, an unsubstituted or substituted phenyl, an unsubstituted or substituted C₁₋₄ aryl alkyl, an unsubstituted or substituted heteroaryl, an unsubstituted or substituted arylamino, an unsubstituted or substituted dialkylamino, an unsubstituted or substituted diarylamino, carboxyalkylamino, carboxydialkylamino, an unsubstituted or substituted tolyl, xylyl, anisyl, mesityl, an unsubstituted or substituted acetoxy, carboxy, an unsubstituted or substituted carboxyethyl, an unsubstituted or substituted alkylcarbonyl, thiol, an unsubstituted or substituted alkylthiol, an unsubstituted or substituted alkyloxy, carboxyamido, an unsubstituted or substituted alkylcarboxyamido, an unsubstituted or substituted dialkylcarboxyamido, an unsubstituted or substituted phenoxy, an unsubstituted or substituted benzyloxy, phenylcarbonyl, benzylcarbonyl, an unsubstituted or substituted nitrophenyl, trialkylsilyl or nitro and the —N(R^{2,} R³) group is cyclized to form a 1,2,3,4-tetrahydroquinolyl (Structure II above or structure III above), piperidinyl (Structure above) or N-substituted-piperizinyl (Structure V above).

This invention provides A compound of structure XI:

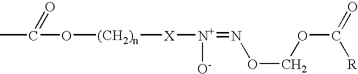

Wherein X is a N-substituted piperizinyl

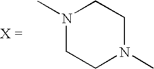

or N- and 4-substituted piperidinyl

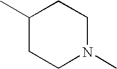

or N-methyl moiety and R is an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted alkoxy, an unsubstituted or substituted morpholino, amino, an unsubstituted or substituted benzyl, an unsubstituted or substituted phenyl, an unsubstituted or substituted C₁₋₄ aryl alkyl, an unsubstituted or substituted heteroaryl, an unsubstituted or substituted arylamino, an unsubstituted or substituted dialkylamino, an unsubstituted or substituted diarylamino, carboxyalkylamino, carboxydialkylamino, an unsubstituted or substituted tolyl, xylyl, anisyl, mesityl, an unsubstituted or substituted acetoxy, carboxy, an unsubstituted or substituted carboxyethyl, an unsubstituted or substituted alkylcarbonyl, an unsubstituted or substituted alkylthiol, an unsubstituted or substituted alkyloxy, carboxyamido, an unsubstituted or substituted alkylcarboxyamido, an unsubstituted or substituted dialkylcarboxyamido, an unsubstituted or substituted phenoxy, an unsubstituted or substituted benzyloxy, phenylcarbonyl, benzylcarbonyl, an unsubstituted or substituted nitrophenyl, trialkylsilyl or nitro.

This invention provides a carbamate compound of structure XII:

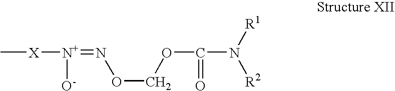

### Structure XII

Wherein X is a N-substituted piperizinyl as in Structure XI, a N- and 4-substituted piperidinyl as in Structure XI or N-methylmoiety and R¹ and R² are each preferentially one of hydrogen, an unsubstituted or substituted C₁₋₁₂ straight chain alkyl, an unsubstituted or substituted C₃₋₁₂ branched chain alkyl, an unsubstituted or substituted C₃₋₁₂ straight chain alkenyl, an unsubstituted or substituted C₃₋₁₂ branched chain alkenyl, an unsubstituted or substituted C₃₋₈ cycloalkyl, an unsubstituted or substituted benzyl, an unsubstituted or substituted phenyl, an unsubstituted or substituted C₁₋₄ aryl alkyl, an unsubstituted or substituted heteroaryl, an unsubstituted or substituted tolyl, xylyl, anisyl, mesityl, an unsubstituted or substituted carboxyethyl, an unsubstituted or substituted alkylcarbonyl, phenylcarbonyl, benzylcarbonyl, an unsubstituted or substituted nitrophenyl, or nitro or the —N(R², R³) group is cyclized to form a 1,2,3,4-tetrahydroquinolyl (Structure II above or structure III above), piperidinyl (Structure IV above), or N-substituted-piperizinyl (Structure V above).

This invention provides a compound O²-(Acetylsalicyloyloxymethyl)-1-(pyrrolidin-1-yl)diazen-1-ium-1,2-diolate as shown in FIG. 6.

This invention provides a compound O²-(Acetylsalicyloyloxymethyl)-1-(N,N-dimethylamino)diazen-1-ium-1,2-diolate as shown in FIG. 6.

This invention provides a compound O²-[2-(4-(Isobutyl)phenyl)propanoyloxymethyl]-1-(pyrrolidin-1-yl)diazen-1-ium-1,2-diolate as shown in FIG. 6.

This invention provides a compound O²-[2-(4-(Isobutyl)phenyl)propanoyloxymethyl]-1-(N,N-dimethylamino)diazen-1-ium-1,2-diolate as shown in FIG. 6.

This invention provides a compound O²-[2-(1-(4-Chlorobenzoyl)-5-methoxy-2-methyl-1 H-indol-3-yl)acetoxymethyl]-1-(pyrrolidin-1-yl)diazen-1-ium-1,2-diolate as shown in FIG. 6.

This invention provides a compound O²-[2-(1-(4-Chlorobenzoyl)-5-methoxy-2-methyl-1H-indol-3-yl)acetoxymethyl]-1-(dimethylamino)diazen-1-ium-1,2-diolate as shown in FIG. 6.

This invention provides a composition comprising an effective amount of one of the compounds described herein in the same molar dose range as recommended for the NSAID from which it was derived.

This invention provides a composition comprising an effective amount of one of the compounds described herein in various dose ranges capable of enhancing therapeutic outcome as recommended for the NSAID from which it was derived.

This invention provides the use of any of the above-mentioned compounds to reduce gastrointestinal side effects of the parent non-steroidal anti-inflammatory drugs (NSAID). The side effects include but are not limited to dyspepsia, nausea and vomiting, abdominal pain, diarrhea, gastric or intestinal bleeding, and gastric and/or intestinal ulceration.

This invention provides the use of any of the above-mentioned compounds for the indications recommended for the unsubstituted NSAID from which it is derived. For example the indication may be pain and inflammation, headache (e.g ibuprofen), cardiovascular protection (e.g. acetylsalicylic acid), rheumatoid or osteoarthritis symptoms (e.g. naproxen, indomethacin), etc.

This invention provides the use of any of the above-mentioned compounds in the same molar dose range as recommended for the NSAID from which it was derived.

This invention provides the use of any of the above-mentioned compounds described in various dose ranges to achieve better therapeutic outcome as recommended for the NSAID from which it was derived.

Exemplification

The invention being generally described, will be more readily understood by reference to the following examples which are included merely for purposes of illustration of certain aspects and embodiments of the present invention, and are not intended to limit the invention.

A group of new NO-releasing non-steroidal anti-inflammatory drugs (NO-NSAIDs), derived from aspirin (O²(Acetylsalicyloyloxymethyl)-1-(pyrrolidin-1-yl)diazen-1-ium-1,2-diolate, 11; O² (Acetylsalicyloyloxymethyl)-1-(N,N-dimethylamino)diazen-1-ium-1,2-diolate, 12), ibuprofen (O²-[2-(4-(Isobutyl)phenyl)propanoyloxymethyl]-1-(pyrrolidin-1-yl)diazen-1-ium-1,2-diolate, 13; O²-[2-(4(Isobutyl)phenyl)propanoyloxymethyl]-1-(N,N-dimethylamino)diazen-1-ium-1,2-diolate, 14) and indomethacin (O²-[2-(1-(4-Chlorobenzoyl)-5-methoxy-2-methyl-1H-indol-3-yl)acetoxymethyl]-1-(pyrrolidin-1-yl)diazen-1-ium-1,2-diolate, 15; O²-[2-(1-(4-Chlorobenzoyl)-5-methoxy-2-methyl-1H-indol-3-yl)acetoxymethyl]-1-(dimethylamino)diazen-1-ium-1,2-diolate, 16) possessing a 1-(pyrrolidin-1-yl)diazen-1-ium-1,2-diolate, or 1-(N,N-dimethylamino)diazen-1-ium-1,2-diolate moiety were synthesized.

Chemistry: O²-Chloromethyl-1-(N,N-dimethylamino)diazen-1-ium-1,2-diolate (9) was prepared according to a modified procedure reported by Tang et al,³³ as illustrated in FIG. 3. Thus, reaction of dimethylamine (6) with nitric oxide gas (40 psi) at room temperature in the presence of sodium methoxide, afforded O²-sodium 1-(N,N-dimethylamino)diazen-1-ium-1,2-diolate (7) in 90% yield. The sodium salt was alkylated with chloromethyl methyl sulfide to afford O²-(methylthiomethyl)-1-(N,N-dimethylamino)diazen-1-ium-1,2-diolate (8), which was subsequently reacted with sulfuryl chloride in dichloromethane for 4 h to afford the O²-chloromethyl-protected diazeniumdiolate 9 in quantitative yield. The target NO-NSAID ester prodrugs 11-16 were synthesized in moderate-to-good yields (40-81%) by condensation of the sodium salt of acetylsalicylic acid, ibuprofen or indomethacin, with O²-chloromethyl intermediates 9 or 10 using the polar aprotic solvent HMPA (FIG. 4).

In vitro COX enzyme inhibition studies, showed that none of these compounds inhibited either the COX-1 or COX-2 isozyme at the highest test compound concentration used (100 μM). See Table 1 below.

TABLE 1
In Vitro COX-1/COX-2 Enzyme Inhibition, and In Vivo
Antiinflammatory Activity Data for NO-NSAIDs 11-16.

| | COX-1 | COX-2 | | AI activity^{c} |
|---|---|---|---|---|
| Compd. | IC₅₀ (μM)^{a} | IC₅₀ (μM)^{a} | COX-2 S.I.^{b} | ID₅₀ (mg/kg) |
| 11 | >100 | >100 | — | 181.8 |
| 12 | >100 | >100 | — | 151.2 |
| 13 | >100 | >100 | — | 66.8 |
| 14 | >100 | >100 | — | 62.3 |
| 15 | >100 | >100 | — | 10.7 |
| 16 | >100 | >100 | — | 5.9 |
| Aspirin | 0.3 | 2.4 | 0.14 | 128.7 |
| Ibuprofen | 2.9 | 1.1 | 2.63 | 67.4 |
| Indomethacin | 0.1 | 5.7 | 0.01 | 4.2 |

^{a}The in vitro test compound concentration required to produce 50% inhibition of COX-1 or COX-2. The result (IC₅₀, μM) is the mean of two determinations acquired using an ovine COX-1/COX-2 assay kit (Catalog No. 560101, Cayman Chemicals Inc., Ann Arbor, MI, USA) and the deviation from the mean is <10% of the mean value.
^{b}Selectivity index (SI) = COX-1 IC₅₀/COX-2 IC₅₀.
^{c}Inhibitory activity in a carrageenan-induced rat paw edema assay. The results are expressed as the ID₅₀ value (mg/kg) at 3 h after oral administration of the test compound.

Thus, attachment of an ester group (the NO-releasing diazeniumdiolate moiety) to the parent NSAID completely abolished the in vitro enzyme inhibitory activity of aspirin, ibuprofen and indomethacin. However, when administered orally to rats, the carrageenan-induced rat paw edema assay (Table 1) provided similar ID₅₀ values to those obtained for the reference drugs. The ibuprofen NO-NSAIDs 13 and 14 showed equipotent anti-inflammatory activities (ID₅₀=66.8 and 62.3 mg/kg respectively) compared to the reference drug ibuprofen (ID₅₀=67.4 mg/kg). Similar results were obtained for the NO-aspirins 11 (ID₅₀=181.8 mg/kg) and 12 (ID₅₀=151.2 mg/kg), and the NO-indomethacin 16 (ID₅₀=5.9 mg/kg), which were 1.1-1.4-fold less potent relative to the parent drugs aspirin (ID₅₀=128.7 mg/kg) and indomethacin (ID₅₀=4.2 mg/kg). In comparison, the NO-indomethacin 15 (ID₅₀=10.7 mg/kg) was about 2.5 fold-less potent than indomethacin. Compounds containing a 1-(pyrrolidin-1-yl)diazen-1-ium-1,2-diolate (11, 13 and 15) moiety were less active than those compounds having a 1-(N,N-dimethylamino)diazen-1-ium-1,2-diolate moiety (12, 14 and 16). It has been reported that aspirin acetylates the Ser530 residue in the COX-1 active site.¹⁴ The observations that both NO-aspirins (11 and 12) were inactive in vitro inhibitors of COX-1 and COX-2 (IC₅₀>100 μM), and that they showed significant anti-inflammatory activities in vivo, strongly suggests that 11 and 12 act as classical prodrugs, which require a metabolic activation reaction (esterase-mediated ester cleavage) to be active. One type of chemical modification used to control the rate of nitric oxide release from diazen-1-ium-1,2-diolates is the attachment of alkyl substituents to the O²-position.³⁴ O²-substituted-diazen-1-ium-1,2-diolates are stable compounds that hydrolyze slowly even in acidic solution.³⁵ Consistent with these observations, when compounds 11-16 were incubated in phosphate buffer solution (PBS) at pH 7.4, the percentage of NO released varied from 14.3 to 16.1% which is indicative of slow NO release. In contrast to recently reported O²-acetoxymethyl-1-(pyrrolidin-1-yl or N,N-diethylamino)diazen-1-ium-1,2-diolates,³⁶ which are stable prodrugs in neutral aqueous media but which released about 1.8 equivalents of NO (>90% release) per mol of drug upon metabolism by porcine liver esterase (PLE), the ester prodrugs 11-16 are hydrolyzed much less extensively (16.3 to 19.2% NO release) . However, the effect of non-specific esterases present in guinea pig serum on the NO release properties of compounds 11-16 was substantially higher (81.6-93.6% range) than that observed (16.3-19.2% range) upon incubation with PLE (see Table 2).

TABLE 2
Nitric Oxide Release Data for NO-NSAIDs 11-16.

| | % of Nitric oxide released^{a} | | |
|---|---|---|---|
| Compd | PBS (pH 7.4)^{b} | PLE^{c} | GP-Serum^{d} |
| 11 | 14.8 ± 0.1 | 18.5 ± 0.1 | 88.9 ± 0.2 |
| 12 | 15.4 ± 0.1 | 19.1 ± 0.1 | 81.6 ± 0.1 |
| 13 | 14.9 ± 0.1 | 16.3 ± 0.1 | 89.2 ± 0.1 |
| 14 | 16.1 ± 0.1 | 17.3 ± 0.1 | 93.6 ± 0.1 |
| 15 | 15.1 ± 0.1 | 16.3 ± 0.1 | 89.1 ± 0.1 |
| 16 | 14.3 ± 0.1 | 16.9 ± 0.1 | 86.3 ± 0.1 |
| 7 | 95.2 ± 0.1 | — | — |
| O²-sodium 1- | 94.0 + 0.1 | — | — |
| (pyrrolidin-1-yl) | | | |
| diazen-1-ium-1,2- | | | |
| diolate | | | |

^{a}Percent of nitric oxide released (± SEM, n = 3) relative to a theoretical maximum release of 2 mol of NO/mol of test compound.
^{b}Incubated in phosphate buffer solution (PBS, pH 7.4) at 37° C. for 1.5 h.
^{c}Incubated in the presence of 2 equivalents of pig liver esterase (based on a ratio of 1 mol of test compound/2 mol of esterase) in phosphate buffer solution (pH 7.4) at 37° C. for 1.5 h.
^{d}Test compound (2.0 × 10⁻⁴ mmol) incubated with guinea pig serum (260 μL) in phosphate buffer solution (pH 7.4) at 37° C. for 1.5 h.

These data indicate the non-specific serum esterases present in guinea pig serum cleave these NO-NSAIDs more effectively than PLE. Although conventional NO donors can protect the stomach against NSAID-induced gastric damage, they do not do so as effectively as NSAIDs (including aspirin) that are chemically linked to an NO-releasing moiety.³⁷ A plausible mechanism for the hydrolysis of these NO-NSAID ester prodrugs 11-16 is presented in FIG. 5. The NO-NSAID ester prodrugs 11-16 were designed with a one-carbon methylene spacer between the carboxy group and the diazen-1-ium-1,2-diolate O²-atom, such that the O²-(hydroxymethyl)diazen-1-ium-1,2-diolate compound formed after ester cleavage would spontaneously eliminate formaldehyde to produce the free NONOate compound that can subsequently fragment to release two molecules of NO.

One of the common side effects of NSAID therapy is gastrointestinal irritation and bleeding. It was therefore essential to evaluate the prodrugs 11-16 ulcerogenicity in comparison to that induced by the three parent drugs. The severity of gastric damage was expressed as an ulcer index (Table 3).

TABLE 3
Gastric ulcer index produced by an acute
administration of the test compounds 11-16 and the reference
drugs aspirin, ibuprofen and indomethacin.

| Compd. | Ulcer index^{a} |
|---|---|
| aspirin | 57.4 ± 3.1^{b} |
| ibuprofen | 45.8 ± 2.9^{b} |
| indomethacin | 34.4 ± 4.2^{c} |
| 11 | 0^{d} |
| 12 | 0^{d} |
| 13 | 0^{e} |
| 14 | 0^{e} |
| 15 | 0.7 ± 0.11^{f} |
| 16 | 3.0 ± 0.3^{f} |
| control group | 0^{g} |

^{a}The average overall length (in mm) of individual ulcers in each stomach ± SEM, n = 4, at 6 h after oral administration of the test compound.
^{b}250 mg/kg dose.
^{c}30 mg/kg dose.
^{d}Equivalent amount to 250 mg of aspirin/kg.
^{e}Equivalent amount to 250 mg of ibuprofen/kg.
^{f}Equivalent amount to 30 mg of indomethacin/kg.
^{g}1.0% methylcellulose solution.

There was a remarkable difference between the ulcer index values for the NO-NSAIDs (UI=0−3.0), and the reference drugs aspirin (UI=57.4, 250 mg/kg po dose), ibuprofen (UI=45.7, 250 mg/kg po dose) and indomethacin (34.4, 30 mg/kg po dose). This UI data suggests a much more safer pharmacological profile for hybrid NO-NSAIDs containing either a 1-(pyrrolidin-1-yl or N,N-dimethylamino)diazen-1-ium-1,2-diolate groups, relative to the parent drugs. No evidence of gastric ulcerogenicity (UI=0) was observed (FIG. 2) for either the NO-aspirin (11, 12) and NO-ibuprofen (13, 14) ester prodrugs. The NO-indomethacin compounds (15, 16) caused minimal ulcerogenicity (UI=0.7-3.0 range).

### Conclusions

Hybrid NO-NSAID ester prodrugs possessing a 1-(pyrrolidin-1-yl)diazen-1-ium-1,2-diolate (11, 13, 15) or 1-(N,N-dimethylamino)diazen-1-ium-1,2-diolate (12, 14, 16), moiety attached via a one-carbon methylene spacer to the carboxylic acid group of traditional NSAIDs constitutes a useful concept for the rational design of anti-inflammatory drugs with reduced gastric side effects (ulcerogenicity). Virtually every NSAID having a free carboxylic acid is suitable for application of this methodology. In vivo activation (hydrolysis) of these NO-NSAIDs by plasma esterases, rather than liver esterases, would be expected to improve the NO release profile compared to that observed for organic nitrates which require a more metabolically demanding three-electron reduction for the release of NO, or a thiol cofactor such as L-cysteine or glutathione required for the release of NO from furoxans. Hybrid NO-aspirins having a diazen-1-ium-1,2-diolate moiety could be a useful alternative to the use of aspirin as an antithrombotic agent (inhibition of platelet aggregation) in the long-term prophylactic prevention of stroke and myocardial infarction.

General. Melting points were recorded with a Thomas-Hoover capillary apparatus and are uncorrected. ¹H NMR spectra were acquired using a Bruker AM-300 spectrometer (300 MHz). Infrared spectra were recorded using a Nicolet IR-500 Series II spectrometer. Silica gel column chromatography was carried out using Merck 7734 (60-200 mesh) silica gel.

Microanalyses were within ±0.4% of theoretical values for all elements listed. See Table 4 below.

TABLE 4
Microanalytical Data

| | Empirical | Calculated | | | Found | | |
|---|---|---|---|---|---|---|---|
| Compound | Formula | C | H | N | C | H | N |
| 11 | C₁₄H₁₇N₃O₆ | 52.01 | 5.30 | 13.00 | 51.99 | 5.28 | 12.90 |
| 12 | C₁₂H₁₅N₃O₆ | 48.48 | 5.09 | 14.14 | 48.78 | 4.97 | 14.01 |
| 13 | C₁₈H₂₇N₃O₄ | 61.87 | 7.79 | 12.03 | 61.83 | 7.79 | 12.03 |
| 14 | C₁₆H₂₅N₃O₄ | 59.42 | 7.79 | 12.99 | 59.41 | 7.80 | 12.89 |
| 15 | C₂₄H₂₅ClN₄O₆ | 57.54 | 5.03 | 11.18 | 57.53 | 5.03 | 11.22 |
| 16 | C₂₂H₂₃ClN₄O₆ | 55.64 | 4.88 | 11.80 | 55.63 | 4.89 | 11.79 |

Acetyl salicylic acid (aspirin), racemic ibuprofen and indomethacin were purchased from the Sigma Chemical Co. O²(chloromethyl)diazen-1-ium-1,2-diolate (10) was prepared according to a literature procedure³³ except that the reaction of O²-sodium 1-(pyrrolidin-1-yl)diazen-1-ium-1,2-diolate with chloromethyl methyl sulfide was carried out in HMPA at 25° C. for 48 h. Nitric oxide gas was purchased from BOC Scientific (Burlington, ON). All other chemicals were purchased from the Aldrich Chemical Co. (Milwaukee, Wis.). The in vivo anti-inflammatory and ulcer index assays were carried out using protocols approved by the Health Sciences Animal Welfare Committee at the University of Alberta.

O²-Sodium 1-(N,N-dimethylamino)diazen-1-ium-1,2-diolate (7). Dimethylamine (6, 4.5 g, 0.1 mol) was added to a solution of sodium methoxide (0.1 mol, 24 mL of a 25% w/v solution in methanol) and diethyl ether (300 mL) with stirring at 25° C. This mixture was flushed with dry nitrogen for five minutes and then the reaction was allowed to proceed under an atmosphere of nitric oxide (40 psi internal pressure) with stirring at 25° C. for 19 h. The product, which precipitated as a fine white powder, was isolated by filtration and then suspended in diethyl ether (100 mL) upon stirring for 15 min. The suspension was filtered, the solid collected was dried at 25° C. under reduced pressure until a constant weight was achieved after about 2 h to afford 7 as a fine white powder (11.5 g, 90%); mp 258-260° C. (dec.); ¹H NMR (DMSO-d₆) δ 2.97 [s, 6H, N(CH₃)₂]. Product 7 was used immediately after drying without further purification for the preparation of compound 8.

O²-(Methylthiomethyl)-1-(N,N-dimethylamino)diazen-1-ium-1,2-diolate (8). The sodium diazeniumdiolate 7 (7 g, 54.6 mmol) was added to a suspension of potassium carbonate (1.5 g, 11 mmol) and HMPA (80 mL) at 4° C. and this mixture was stirred for 30 min. Chloromethyl methyl sulfide (6.3 g, 65.6 mmol) was added drop wise, and the reaction was allowed to proceed at 25° C. for 72 h with stirring. Ethyl acetate (200 mL) was added to quench the reaction, the solids were filtered off and the organic phase was washed with water (5×80 mL), dried (Na₂SO₄), and solvent was removed in vacuo to give a liquid residue which was purified by silica gel column chromatography using EtOAc-hexane (1:4, v/v) as eluent. Compound 8 (1.97 g, 21%) was obtained as a pale yellow liquid; ¹H NMR (CDC₁₃) δ 2.24 (s, 3H, SCH₃), 3.01 [s, 6H, N(CH₃)₂], 5.21 (s, 2H, OCH₂S). Compound 8 was used immediately for the subsequent preparation of the O²-chloromethyl derivative 9.

O²-(Chloromethyl)-1-(N,N-dimethylamino)diazen-1-ium-1,2-diolate (9). A solution of compound 8 (1.8 g, 11.4 mmol) in dichloromethane (20 mL) was cooled to 4° C., sulfuryl chloride (2.3 g, 17.1 mmol, 17 mL of a 1.0 M solution in dichloromethane) was added drop wise, the ice bath was removed and the reaction mixture was stirred at 25° C. for 3 h. The brown solid suspended in the reaction media was removed by filtration, and the solvent was evaporated to furnish 9 (1.7 g, quantitative yield); ¹H NMR (CDC₁₃) δ 3.01 [s, 6H, N(CH₃)₂], 5.76 (s, 2H, ClCH₂O) Compound 9 was used without further purification for the synthesis of products 12, 14 and 16.

General Method for the Preparation of NO-NSAIDs (11-16). Sodium carboxylates of the respective NSAID (aspirin, ibuprofen or indomethacin) were prepared in situ by stirring each acid (5 mmol) in a suspension of sodium carbonate (0.53 g, 5 mmol) and HMPA (7 mL) for 19 h at 25° C. A solution of a O²-(chloromethyl)diazen-1-ium-1,2-diolate 9 or 10 (5 mmol) in HMPA (3 mL) was then added, and the reaction was allowed to proceed for 24 h at 25° C. Ethyl acetate (60 mL) was added, the mixture was washed with water (5×30 mL), the organic phase was dried (Na₂SO₄), and the solvent was removed in vacuo. The residue obtained was purified by silica gel column chromatography using CHCl₃-EtOAc-hexane (35:15:50, v/v/v) as eluent for compounds 11, 12, 15, and 16; EtOAc-hexane (1:4, v/v) for compound 13; and hexane-ether (3:1, v/v) for compound 14. Physical and spectral data for 11-16 are listed below.

O²(Acetylsalicyloyloxymethyl)-1-(pyrrolidin-1-yl)diazen-1-ium-1,2-diolate (11). 46% yield; white crystals; mp 110-112° C.; IR (CHCl₃) 3019 (C—H aromatic), 2992 (C—H aliphatic), 1770 (CO₂), 1736 (CO₂), 1259, 1199 (N═N—O) cm⁻¹; ¹H NMR (CDCl₃) δ 1.95 (quintet, J=6.9 Hz, 4H, pyrrolidinyl H-3, H-4), 2.34 (s, 3H, COCH₃), 3.57 (t, J=6.9 Hz, 4H, pyrrolidinyl H-2, H-5), 5.97 (s, 2H, OCH₂O), 7.12 (d, J=8.1 Hz, phenyl H-3), 7.34 (t, J=8.1 Hz, phenyl H-5), 7.60 (td, J=8.1, 1.5 Hz, phenyl H-4), 8.08 (dd, J=8.1, 1.5 Hz, phenyl H-6). Anal. (C₁₄H₁₇N₃O₆) C, H, N.

O²(Acetylsalicyloyloxymethyl)-1-(N,N-dimethylamino)diazen-1-ium-1,2-diolate (12). 40% yield; white crystals; mp 88-89° C.; IR (KBr) 3019 (C—H aromatic), 2979 (C—H aliphatic), 1756 (CO₂), 1609 (CO₂), 1219, 1184 (N═N—O) cm⁻¹; ¹H NMR (CDCl₃) δ 2.34 (s, 3H, COCH₃), 3.07 [s, 6H, N(CH₃)₂], 6.02 (s, 2H, OCH₂O), 7.12 (d, J=8.1 Hz, phenyl H-3), 7.34 (t, J=8.1 Hz, phenyl H-5), 7.60 (td, J=8.1, 1.5 Hz, phenyl H-4), 8.07 (dd, J=8.1, 1.5 Hz, phenyl H-6). Anal. (C₁₂H₁₅N₃O₆) C, H, N.

O²-[2-(4-(Isobutyl)phenyl)propanoyloxymethyl]-1-(pyrrolidin-1-yl)diazen-1-ium-1,2-diolate (13). 58% yield; yellow oil; IR (KBr) 2985 (C—H aromatic), 2864 (C—H aliphatic), 1750 (CO₂), 1286, 1129 (N═N—O) cm⁻¹; ¹H NMR (CDCl₃) δ 0.89 [d, J=6.6 Hz, 6H, CH(CH₃)₂], 1.50 (d, J=7.2 Hz, 3H, PhCHCH₃), 1.79-1.89 [m, 1H, CH(CH₃)₂], 1.91-1.94 (m, 4H, pyrrolidinyl H-3, H-4), 2.43 (d, J=7.2 Hz, 2H, PhCH₂CH), 3.45-3.50 (m, 4H, pyrrolidinyl H-2, H-5), 3.73 (q, J=7.2 Hz, 1H, PhCHCH₃), 5.71 (d, J=7.2 Hz, 1H, OCH′HO), 5.77 (d, J=7.2 Hz, 1H, OCH′HO), 7.07 (d, J=7.8 Hz, 2H, phenyl H-3, H-5), 7.19 (d, J=7.8 Hz, 2H, phenyl H-2, H6). Anal. (C₁₈H₂₇N₃O₄) C, H, N.

O²-[2-(4-(Isobutyl)phenyl)propanoyloxymethyl]-1-(N,N-dimethylamino)diazen-1-ium-1,2-diolate (14). 81% yield; yellow oil; IR (KBr) 2959 (C—H aromatic), 2871 (C—H aliphatic), 1763 (CO₂), 1279, 1138 (N═N—O) cm⁻¹; ¹H NMR (CDC₁₃) δ 0.89 [d, J=6.9 Hz, 6H, CH(CH₃)₂], 1.50 (d, J=6.9 Hz, 3H, PhCHCH₃), 1.83 [septet, J=6.9 Hz, 1H, CH(CH₃)₂], 2.43 (d, J=6.9 Hz, 2H, PhCH₂CH), 2.97 [s, 6H, N(CH₃)₂], 3.74 (q, J=6.9 Hz, 1H, PhCHCH₃), 5.74 (d, J=7.2 Hz, 1H, OCH′HO), 5.79 (d, J=7.2 Hz, 1H, OCH′HO), 7.08 (d, J=7.8 Hz, 2H, phenyl H-3, H-5), 7.19 (d, J=7.8 Hz, 2H, phenyl H-2, H6) . Anal. (C₁₆H₂₅N₃O₄) C, H, N.

O²-[2-(1-(4-Chlorobenzoyl)-5-methoxy-2-methyl-1H-indol-3-yl)acetoxymethyl]-1-(pyrrolidin-1-yl)diazen-1-ium-1,2-diolate (15). 51% yield; yellow oil; IR (KBr) 3019 (C—H aromatic), 2979, 2885 (C—H aliphatic), 1756 (CON), 1689 (CO₂), 1293, 1165 (N═N—O) cm⁻¹; ¹H NMR (CDCl₃) δ 1.88 (quintet, J=6.9 Hz, 4H, pyrrolidinyl H-3, H-4), 2.36 (s, 3H, CH₃), 3.40 (t, J=6.9 Hz, 4H, pyrrolidinyl H-2, H-5), 3.71 (s, 2H, CH₂CO₂), 3.83 (s, 3H, OCH₃), 5.77 (s, 2H, OCH₂O), 6.66 (dd, J=9, 2.4 Hz, 1H, indolyl H-6), 6.90 (d, J=9 Hz, 1H, indolyl H-7), 6.94 (d, J=2.4 Hz, indolyl H-4), 7.47 (d, J=8.7 Hz, 2H, benzoyl H-3, H-5), 7.65 (d, J=8.7 Hz, 2H, benzoyl H-2, H-6) . Anal. (C₂₄H₂₅ClN₄O₆) C, H, N.

O²-[2-(1-(4-Chlorobenzoyl)-5-methoxy-2-methyl-1H-indol-3-yl)acetoxymethyl]-1-(dimethylamino)diazen-1-ium-1,2-diolate (16). 69% yield; yellow oil; IR (KBr) 2979, 2925 (C—H aliphatic), 1763 (CON), 1689 (CO₂), 1333, 1064 (N═N—O) cm⁻¹; ¹H NMR (CDC₁₃) δ 2.35 (s, 3H, CH₃), 2.94 [s, 6H, N(CH₃)₂], 3.71 (s, 2H, CH₂CO₂), 3.81 (s, 3H, OCH₃), 5.80 (s, 2H, OCH₂O), 6.66 (dd, J=8.7, 2.4 Hz, 1H, indolyl H-6), 6.88 (d, J=8.7 Hz, 1H, indolyl H-7), 6.93 (d, J=2.4 Hz, 1H, indolyl H-4), 7.46 (d, J=8.4 Hz, 2H, benzoyl H-3, H-5), 7.64 (d, J=8.4, 2H, benzoyl H-2, H-6). Anal. (C₂₂H₂₃ClN₄O₆) C, H, N.

Cyclooxygenase Inhibition Studies. The ability of the test compounds listed in Table 1 to inhibit ovine COX-1 and COX-2 (IC₅₀ value, μM) was determined using an enzyme immuno assay (EIA) kit (catalog no. 560101, Cayman Chemical, Ann Arbor, Mich., USA) according to our previously reported method.³⁸

Anti-inflammatory Assay. The test compounds 11-16 and the reference drugs (aspirin, ibuprofen and indomethacin) were evaluated using the in vivo rat carrageenan-induced foot paw edema model reported previously.^{39,40}

Nitric Oxide Release Assay: In vitro nitric oxide release, upon incubation with phosphate buffer, pig liver esterase, or guinea pig serum, was determined by quantification of nitrite produced by the reaction of nitric oxide with oxygen and water using the Griess reaction. Nitric oxide release data were acquired for test compounds (11-16), and the reference compounds O²-sodium 1-(pyrrolidin-1-yl)dizen-1-ium-1,2-diolate, and O²-sodium 1-(N,N-dimethylamino)diazen-1-ium-1,2-diolate (7) using the reported procedures.⁴¹

Acute Ulcerogenesis Assay: The ability to produce gastric damage was evaluated according to a reported procedure.⁴² Ulcerogenic activity was evaluated after oral administration of aspirin (250 mg/kg), ibuprofen (250 mg/kg), indomethacin (30 mg/kg) or an equivalent amount of the correspondent test compound (11-16). All drugs were suspended and administered in 1.7 mL of a 1% methylcellulose solution. Control rats received oral administration of vehicle (1.7 mL of 1.0% methylcellulose solution). Food, but not water, was removed 24 h before administration of test compounds. Six hours after oral administration of the drug, rats were euthanized in a CO₂ chamber and their stomachs were removed, cut out along the greater curvature of the stomach, gently rinsed with water and placed on ice. The number and the length of ulcers were determined using a magnifier lens. The severity of the gastric lesion was measured along its greatest length (1 mm=rating of 1, 1-2 mm=rating of 2, >2 mm=rating according to their length in mm). The average overall length (in mm) of individual ulcers in each tissue was designated as the “ulcer index”. Each experimental group consisted of four rats.

### REFERENCES

(1) Brooks, P. M.; Day, R. O. Nonsteroidal anti-inflammatory drugs-differences and similarities. N. Engl. J. Med. 1991, 324, 1716-1725.
(2) Cathella-Lawson, F.; Reilly, M. P.; Kapoor, S. C. Cyclooxygenase inhibitors and the antiplatelet effects of aspirin. N. Engl. J. Med. 2001, 345, 1809-1817.
(3) Vane, J.; Botting, R. M. Mechanism of action of nonsteroidal anti-inflammatory drugs. Am. J. Med. 1998, 104, (Suppl 3A), 2S-8S.
(4) Rodriguez, L. A. The effect of NSAIDs on the risk of coronary heart disease: fusion of clinical pharmacology and pharmacoepidemiologic data. Clin. Exp. Rheumatol. 2001, 19, (Suppl 25), S41-S44.
(5) Schoen, R. T.; Vender, R. J. Mechanisms of nonsteroidal anti-inflammatory drugs gastric damage. Am. J. Med. 1989, 86, 449-457.
(6) Hollander, D. Gastrointestinal complications of nonsteroidal anti-inflammatory drugs: prophylactic and therapeutic strategies. Am. J. Med. 1994, 96, 274-281.
(7) Rainsford, K. D. Profile and mechanisms of gastrointestinal and other side effects of nonsteroidal anti-inflammatory drugs (NSAIDs). Am. J. Med. 1999, 107, 27S-36S.
(8) Soll, A. H.; Weinstein, W. M.; Kurata, J.; McCarthy, D. Nonsteroidal anti-inflammatory drugs and peptic ulcer disease. Ann. Intern. Med. 1991, 114, 307-319.
(9) Bjarnason, I.; Hayllar, J.; MacPherson, A. J.; Russell, A. S. Side effects of nonsteroidal anti-inflammatory drugs on the small and large intestine in humans. Gastroenterology 1993, 104, 1832-1847.
(10) Wallace, J. L.; Soldato, P. D. The therapeutic potential of NO-NSAIDs. Fund. Clin. Pharmacol. 2003, 17, 11-20.
(11) Sanmuganathan, P. S.; Ghahramani, P.; Jackson, P. R. Aspirin for primary prevention of coronary heart disease: safety and absolute benefit related to coronary risk derived from meta-analysis of randomized trials. Heart 85, 265-271.
(12) Antiplatelet Trialist's Collaboration. Collaborative overview of randomized trials of antiplatelet therapy-I: prevention of death, myocardial infarction, and stroke by prolonged antiplatelet therapy in various categories of patients. BMJ 1994, 308, (81-106).
(13) Meade, E. A.; Smith, W. L.; DeWitt, D. L. Differential Inhibition of Prostaglandin Endoperoxide Synthase (Cyclooxygenase) Isozymes by Aspirin and Other Non-steroidal Anti-inflammatory Drugs. J. Biol. Chem. 1993, 268, 6610-6614.
(14) Awtry, E. H.; Loscalzo, J. Cardiovascular drugs: aspirin. Circulation 2000, 101, 1206-1218.
(15) Catella-Lawson, F.; Crofford, L. J. Cyclooxygenase inhibition and thrombogenicity. Am. J. Med. 2001, 110, (Suppl 3A), 28-32S.
(16) Hamberg, M.; Seensson, J.; Samuelson, B. Thromboxanes: a new group of biologically active compounds derived from prostaglandin endoperoxidases. Proc. Natl. Acad. Sci. USA, 1975, 72, 2294-2298.
(17) Lee, M.; Cryer, B. Dose effects of aspirin on gastric prostaglandins and stomach mucosal injury. Ann. Intern. Med. 1994, 120, 184-189.
(18) Meade, T. W.; Roderick, P. J.; Brennan, P. J.; Wilkes, H. C.; Kelleher, C. C. Extracraneal bleeding and other symptoms due to low dose aspirin and low intensity oral anticoagulation. Throm. Haemost. 1992, 68, 1-6.
(19) Lanas, A. Nitrovasodilators, low-dose aspirin, other non-steroidal anti-inflammatory drugs, and the risk of upper gastrointestinal bleeding. N. Engl. J. Med. 2000, 343, 834-839.
(20) Lanza, F. L. A review of gastric ulcer and gastroduodenal injury in normal volunteers receiving aspirin and other nonsteroidal anti-inflammatory drugs. Scand. J. Gastroenterol. 1989, 24, (Suppl. 163), 24-31.
(21) Rainsford, K. D.; Willis, C. Relationship of gastric mucosal damage induced in pigs by anti-inflammatory drugs to their effects on prostaglandin production. Digest. Dis. Sci. 1982, 27, 624-635.
(22) Wallace, J.; McCafferty, D.; Carter, L.; McKnight, W.; Argentieri, D. Tissue selective inhibition of prostaglandin synthesis in rat by tepoxalin: anti-inflammatory without gastropathy. Gastroenterology 1993, 105, 1630-1636.
(23) Whittle, B. Temporal relationship between cyclooxygenase inhibition, as measured by prostacyclin biosynthesis, and the gastrointestinal damage induced by indomethacin in the rat. Gastroenterology 1981, 80, 94-98.
(24) MacNaughton, W. K.; Cirino, G.; Wallace, J. Endothelium-derived relaxing factor (nitric oxide) has protective actions in the stomach. Life Sci. 1989, 45, 1869-1876.
(25) Kitagawa, H.; Takeda, F.; Kohei, H. Effect of endothelium-derived relaxing factor on the gastric lesion induced by HCl in rats. J. Pharmacol. Exp. Ther. 1990, 253, 1133-1137.
(26) Wallace, J.; Reuter, B.; Cicala, C.; McKnight, W.; Grisham, M.; Cirino, G. Novel nonsteroidal anti-inflammatory drug derivatives with markedly reduced ulcerogenic properties in the rat. Gastroenterology 1994, 107, 173-179.
(27) Wallace, J.; Reuter, B.; Cicala, C.; McKnight, W.; Grisham, M.; Cirino, G. A Diclofenac derivative without ulcerogenic properties. Eur. J. Pharmacol. 1994, 257, 249-255.
(28) Reuter, B.; Cirino, G.; Wallace, J. Markedly reduced intestinal toxicity of a diclofenac derivative. Life Sci. 1994, 55, PL1-PL8.
(29) Cuzzolin, L.; Conforti, A.; Adami, A. Anti-inflammatory potency and gastrointestinal toxicity of a new compound. NO-Naproxen. Pharmacol. Res. 1995, 31, 61-65.
(30) Davies, N. M. NO-naproxen vs naproxen: ulcerogenic, analgesic and anti-inflammatory effects. Aliment. Pharmacol. Ther. 1997, 11, 69-79.
(31) Csont, T.; Ferdinandy, P. Cardioprotective effects of glyceryl trinitrate: beyond vascular nitrate tolerance. Pharmacol. Ther. 2005, 105, 57-68.
(32) Keefer, L. K. Progress toward the clinical application of the nitric oxide-releasing diazeniumdiolates. Annu. Rev. Pharmacol. Toxicol. 2003, 43, 585-607.
(33) Tang, X.; Xian, M.; Trikha, M.; Honn, K. V.; Wang, P. G. Synthesis of peptide-diazeniumdiolate conjugates: towards enzyme activated antitumor agents. Tetrahedron Lett. 2001, 42, 2625-2629.
(34) Saavedra, J. E.; Dunams, T. M.; Flippen-Anderson, J. L.; Keefer, L. K. Secondary amine/nitric oxide complex ions, R2N[N(O)NO]-O-functionalization chemistry. J. Org. Chem. 1992, 57, 6134-6138.
(35) Hrabie, J. A.; Klose, J. R. New nitric oxide-releasing zwitterions derived from polyamines. J. Org. Chem. 1993, 58, 1472-1476.
(36) Saavedra, J. E.; Shami, P. J.; Wang, L. Y.; Davies, K. M.; Bohth, M. N.; Citro, M. L.; Keefer, L. K. Esterase-sensitive nitric oxide donors of the diazeniumdiolate family: in vitro antileukemic activity. J. Med. Chem. 2000, 43, 261-269.
(37) Wallace, J.; Reuter, B.; Cirino, G. Nitric oxide-releasing non-steroidal anti-inflammatory drugs: a novel approach for reducing gastrointestinal toxicity. J. Gastroenterol. Hepatol. 1994, 9, S40-S44.
(38) Rao, P. N. P.; Amini, M.; Li, H.; Habeeb, A.; Knaus, E. E. Design, synthesis, and biological evaluation of 6-substituted-3-(4-methanesulfonylphenyl)-4-phenylpyran-2-ones: a novel class of diarylheterocyclic selective cyclooxygenase-2 inhibitors. J. Med. Chem. 2003, 46, 4872-4882.
(39) Winter, C. A.; Risley, E. A.; Nuss, G. W.; Carrageenan-induced edema in hind paw of the rat as an assay for anti-inflammatory drugs. Proc. Soc. Exp. Biol. Med. 1962, 111, 544-552.
(40) Kumar, P.; Knaus, E. E. Synthesis and anti-inflammatory activity of N-substituted dihydropyridylacetic acids, esters and amides. Drug Design Delivery 1987, 2, 145-149.
(41) Velázquez, C.; Vo, D.; Knaus, E. E. Syntheses, calcium channel modulation effects, and nitric oxide release studies of O2-alkyl-1-(pyrrolidin-1-yl)diazen-1-ium-1,2-diolate 4-aryl(heteroaryl)-1,4-dihydro-2,6-dimethyl-3-nitropyridine-5-carboxylates. Drug Dev. Res. 2003, 60, 204-216.
(42) Cocco, M. T.; Congiu, C.; Onnis, V.; Morelli, M.; Felipob, V.; Caulia, O. Synthesis of new 2-arylamino-6-trifluoromethylpyridine-3-carboxylic acid derivatives and investigation of their analgesic activity. Bioorg. Med. Chem. 2004, 12, 4169-4177.

1. A compound of the formula: 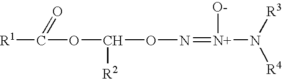
wherein R¹ is an uncarboxylated core of a non-steroidal anti-inflammatory drug, R² is selected from the group consisting of a hydrogen, a C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, an alkoxy, a nitrile, a halo, a morpholino, an amino, a benzyl, a phenyl, a C₁₋₄ aryl alkyl, a heteroaryl, an arylamino, a dialkylamino, a diarylamino, a carboxyalkylamino, a carboxydialkylamino, a tolyl, a xylyl, an anisyl, a mesityl, an acetoxy, a carboxy, a carboxyethyl, an alkylcarbonyl, a thiol, an alkylthiol, an alkyloxy, a carboxyamido, an alkylcarboxyamido, a dialkylcarboxyamido, a phenoxy, a benzyloxy, a phenylcarbonyl, a benzylcarbonyl, a nitrophenyl, a trialkylsilyl and a nitro;
R³ and R⁴ are selected from the group consisting of C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, a morpholino, an amino, a benzyl, a C₁₋₄ aryl alkyl.

2. The compound of claim 1, wherein R³ and R⁴ are same or different from each other.

3. The compound of claim 1, wherein the R² group is substituted or unsubstituted.

4. The compound of claim 1, wherein the —N(R³, R⁴) group is cyclized to form a structure selected from the group consisting of a 1,2,3,4-tetrahydroquinolyl 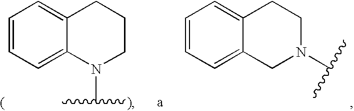
a piperidinyl 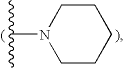
and a N-substituted-piperizinyl

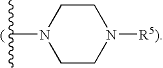

5. The compound of claim 4, wherein R⁵ is selected from the group consisting of a C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, a benzyl, a phenyl, a C₁₋₄ aryl alkyl, a heteroaryl, a tolyl, a xylyl, an anisyl, a mesityl, a carboxyethyl, an alkylcarbonyl, a phenylcarbonyl, a benzylcarbonyl, a nitrophenyl, and a trialkylsilyl.

6. The compound of claim 5, wherein the R5 group is substituted or unsubstituted.

7. The compound of claim 1, wherein the non-steroidal anti-inflammatory drug is selected from the group consisting of acetylsalicylic acid, ibuprofen, naproxen, indomethacin, salicylic acid, diflunisal, salsalate, olsalazine, sulfasalazine, sulindac, etodolac, mefenamic acid, meclofenamic acid, tolmetin, ketorolac, diclofenac, fenoprofen, ketoprofen, oxaprozin, carprofen, flurbiprofen, nabumetone, other related carboxylic acids with anti-inflammatory activity, and their pharmaceutically suitable salts.

8. A compound of the formula: 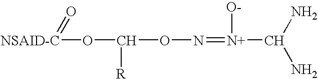
wherein R is same as the R2 in the compound of claim 1, and the compound includes pharmaceutically suitable alkali metal salts or hydrochloride salts thereof.

9. A compound of the formula: 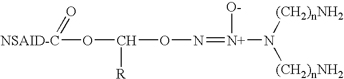
wherein R is same as the R2 in the compound of claim 1, n=1−8, and the compound includes pharmaceutically suitable alkali metal salts or hydrochloride salts thereof.

10. A compound of the formula: 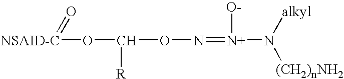
wherein R is same as the R2 in the compound of claim 1, n=1−8, and the compound includes pharmaceutically suitable alkali metal salts or hydrochloride salts thereof.

11. A compound of the formula: 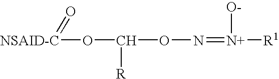
wherein R is same as the R2 in the compound of claim 1, and R1 is a N-substituted amino acid moiety.

12. The compound of claim 11, wherein the N-substituted amino acid moiety is: 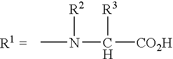
wherein R2 is selected from the group consisting of a hydrogen, a C1-12 straight chain alkyl, a C3-12 branched chain alkyl, a C3-12 straight chain alkenyl, a C3-12 branched chain alkenyl, a C3-8 cycloalkyl, a benzyl, a phenyl, a C1-4 aryl alkyl, a heteroaryl, a tolyl, a xylyl, an anisyl, a mesityl, a carboxyethyl,
and R3 is selected from the group consisting of a hydrogen, a C1-12 straight chain alkyl, a C3-12 branched chain alkyl, a C3-12 straight chain alkenyl, a C3-12 branched chain alkenyl, a C3-8 cycloalkyl, an alkoxy, a nitrile, a halo, a morpholino, an amino, a benzyl, a phenyl, a C1-4 aryl alkyl, a heteroaryl, an arylamino, a dialkylamino, a diarylamino, a carboxyalkylamino, a carboxydialkylamino, a tolyl, a xylyl, an anisyl, a mesityl, an acetoxy, a carboxy, a carboxyethyl, an alkylcarbonyl, a thiol, an alkylthiol, an alkyloxy, a carboxyamido, an alkylcarboxyamido, a dialkylcarboxyamido, a phenoxy, a benzyloxy, a phenylcarbonyl, a benzylcarbonyl, a nitrophenyl, a trialkylsilyl, and a nitro.

13. The compound of claim 12, wherein the N-substituted amino acid moiety is selected from the group consisting of N-methylglycine, N-methylalanine, N-methylphenylalanine, N-methylserine, and any other N-alkyl amino acid.

14. The compound of claim 12, wherein the R2 and R3 are substituted or unsubstituted.

15. An amide bioisostere ester compound of the formula: 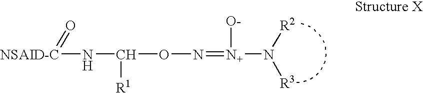
wherein R1 is selected from the group consisting of a hydrogen, a C1-12 straight chain alkyl, a C3-12 branched chain alkyl, a C3-12 straight chain alkenyl, a C3-12 branched chain alkenyl, a C3-8 cycloalkyl, an alkoxy, a nitrile, a halo, a morpholino, an amino, a benzyl, a phenyl, a C1-4 aryl alkyl, a heteroaryl, an arylamino, a dialkylamino, a diarylamino, a carboxyalkylamino, a carboxydialkylamino, a tolyl, a xylyl, an anisyl, a mesityl, an acetoxy, a carboxy, a carboxyethyl, an alkylcarbonyl, a thiol, an alkylthiol, an alkyloxy, a carboxyamido, an alkylcarboxyamido, a dialkylcarboxyamido, a phenoxy, a benzyloxy, a phenylcarbonyl, a benzylcarbonyl, a nitrophenyl, a trialkylsilyl, and a nitro, and the —N(R2, R3) group is cyclized to form a structure selected from the group consisting of a 1,2,3,4-tetrahydroquinolyl 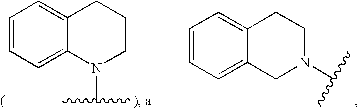
a piperidinyl 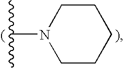
and a N-substituted-piperizinyl

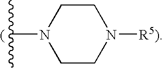

16. The compound of claim 15, wherein R⁵ is selected from the group consisting of a C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, a benzyl, a phenyl, a C₁₋₄ aryl alkyl, a heteroaryl, a tolyl, a xylyl, an anisyl, a mesityl, a carboxyethyl, an alkylcarbonyl, a phenylcarbonyl, a benzylcarbonyl, a nitrophenyl, and a trialkylsilyl.

17. The compound of claim 16, wherein the R5 group is substituted or unsubstituted.

18. The compound of claim 15, wherein the R1 group is substituted or unsubstituted.

19. A compound of the formula: 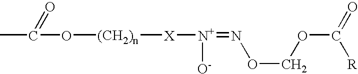
wherein X is selected from the group consisting of N-substituted 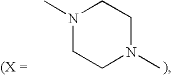
piperizinyl N- and 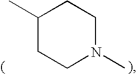
4-substituted piperidinyl and a N-methyl moiety, and
R is selected from the group consisting of a C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, an alkoxy, a nitrile, a halo, a morpholino, an amino, a benzyl, a phenyl, a C₁₋₄ aryl alkyl, a heteroaryl, an arylamino, a dialkylamino, a diarylamino, a carboxyalkylamino, a carboxydialkylamino, a tolyl, a xylyl, an anisyl, a mesityl, an acetoxy, a carboxy, a carboxyethyl, an alkylcarbonyl, an alkylthiol, an alkyloxy, a carboxyamido, an alkylcarboxyamido, a dialkylcarboxyamido, a phenoxy, a benzyloxy, a phenylcarbonyl, a benzylcarbonyl, a nitrophenyl, a trialkylsilyl, and a nitro.

20. The compound of claim 19, wherein the R group is substituted or unsubstituted.

21. A carbamate compound of the formula: 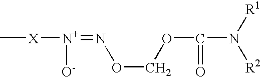
wherein X is selected from the group consisting of N-substituted piperizinyl 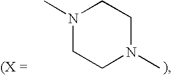
N- and 4-substituted piperidinyl 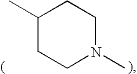
and a N-methyl moiety, and
R¹ and R² are selected from the group consisting of a hydrogen, a C₁₋₁₂ straight chain alkyl, a C₃₋₁₂ branched chain alkyl, a C₃₋₁₂ straight chain alkenyl, a C₃₋₁₂ branched chain alkenyl, a C₃₋₈ cycloalkyl, a benzyl, a phenyl, a C₁₋₄ aryl alkyl, a heteroaryl, a tolyl, a xylyl, an anisyl, a mesityl, a carboxyethyl, an alkylcarbonyl, a phenylcarbonyl, a benzylcarbonyl, a nitrophenyl, a trialkylsilyl, and a nitro.

22. The compound of claim 21, wherein R1 and R2 are substituted or unsubstituted.

23. A compound O²-(Acetylsalicyloyloxymethyl)-1-(pyrrolidin-1-yl)diazen-1-ium-1,2-diolate as shown in FIG. 6.

24. A compound O²-(Acetylsalicyloyloxymethyl)-1-(N,N-dimethylamino)diazen-1-ium-1,2-diolate as shown in FIG. 6.

25. A compound O²-[2-(4-(Isobutyl)phenyl)propanoyloxymethyl]-1-(pyrrolidin-1-yl)diazen-1-ium-1,2-diolate as shown in FIG. 6.

26. A compound O²-[2-(4-(Isobutyl)phenyl)propanoyloxymethyl]-1-(N,N-dimethylamino)diazen-1-ium-1,2-diolate as shown in FIG. 6.

27. A compound O²-[2-(1-(4-Chlorobenzoyl)-5-methoxy-2-methyl-1H-indol-3-yl)acetoxymethyl]-1-(pyrrolidin-1-yl)diazen-1-ium-1,2-diolate as shown in FIG. 6.

28. A compound O²-[2-(1-(4-Chlorobenzoyl)-5-methoxy-2-methyl-1H-indol-3-yl)acetoxymethyl]-1-(dimethylamino)diazen-1-ium-1,2-diolate as shown in FIG. 6.

29. A composition comprising an effective amount of one of the compounds of claim 1 in the same molar dose range as recommended for the NSAID from which it was derived.

30. A composition comprising an effective amount of one of the compounds of claim 1 in various dose ranges capable of enhancing therapeutic outcome as recommended for the NSAID from which it was derived.

31. Use of one of the compounds of claim 1 for reducing gastrointestinal side effects of a parent non-steroidal anti-inflammatory drugs in a subject.

32. Use of one of the compounds of claim 1 in the manufacture of medicament for reducing gastrointestinal side effects of a parent non-steroidal anti-inflammatory drugs in a subject.

33. The use of claim 31, wherein the side effects are selected from the group consisting of dyspepsia, nausea, vomiting, abdominal pain, diarrhea, gastric bleeding, intestinal bleeding, gastric ulceration, and intestinal ulceration.

34. Use of one of the compounds of claim 1 for the indications recommended for the unsubstituted NSAID from which it is derived.

35. Use of one of the compounds of claim 1 in the manufacture of medicament for the indications recommended for the unsubstituted NSAID from which it is derived.

36. The use of claim 34, wherein the indication is selected from the group consisting of pain, inflammation, and headache.

37. The use of claim 36, wherein the unsubstituted NSAID is ibuprofen.

38. The use of claim 34, wherein the indication is cardiovascular protection.

39. The use of claim 38, wherein the unsubstituted NSAID is acetylsalicylic acid.

40. The use of claim 34, wherein the indication is rheumatoid or osteoarthritis symptoms.

41. The use of claim 40, wherein the unsubstituted NSAID is naproxen or indomethacin
